(19) United States
(12) Patent Application Publication
(10) Pub. No.: US 2013/0064878 A1
(43) Pub. Date: Mar. 14, 2013

(54) WOUND HEALING COMPOSITIONS AND METHODS

(71) Applicants: Mayland Chang, Granger, IN (US); Mark A. Suckow, Notre Dame, IN (US); Shahriar Mobashery, Granger, IN (US)

(72) Inventors: Mayland Chang, Granger, IN (US); Mark A. Suckow, Notre Dame, IN (US); Shahriar Mobashery, Granger, IN (US)

(73) Assignee: UNIVERSITY OF NOTRE DAME DU LAC, Notre Dame, IN (US)

(21) Appl. No.: 13/584,653

(22) Filed: Aug. 13, 2012

Related U.S. Application Data
(60) Provisional application No. 61/522,544, filed on Aug. 11, 2011.
(60) Provisional application No. 61/522,554, filed on Aug. 11, 2011.

(51) Int. Cl. A61K 31/38; A61K 9/70; A61P 17/02

(52) U.S. Cl. 424/445; 514/430

(57) ABSTRACT

The invention provides a method of accelerating the healing process of a skin or subdermal wound. The method can include administering to a mammal afflicted with a skin or subdermal wound an effective amount of a gelatinase inhibitor, or a pharmaceutically acceptable salt thereof, wherein the gelatinase inhibitor is effective to accelerate the healing process of the skin wound. The method is particularly effective when the mammal is suffering from diabetes. The gelatinase inhibitor can be topically administered, for example, in the form of a cream, gel, lotion, ointment, salve, or solution.

### CROSS REFERENCE TO RELATED APPLICATIONS

This application claims benefit of U.S. Provisional Application No. 61/522,544, filed 11 Aug. 2011, and also claims benefit of U.S. Provisional Application No. 61/522,554, filed 11 Aug. 2011, and which applications are incorporated herein by reference. A claim of priority to all is made.

### GOVERNMENT SUPPORT

This invention was made with government support under Grant No. CA122417 awarded by the National Institutes of Health. The Government has certain rights in the invention.

### BACKGROUND OF THE INVENTION

Diabetes mellitus is a complex metabolic disease that affects more than 340 million individuals in the world, including 25.8 million Americans. Diabetic patients have impaired ability to metabolize glucose, and the ensuing hyperglycemia results in many complications, which include damage to the vasculature and the inability to heal wounds. The vascular damage in diabetes results in ischemia as a contributing factor to the persistence of wounds, causing inflammation and triggering production of reactive oxygen species, which prevent wound closure by damaging the extracellular matrix (ECM). Matrix metalloproteinases (MMPs), a family of 26 zinc-dependent endopeptidases, normally restructure the ECM in an effort to repair the wound, but the ischemic condition in diabetic wounds presents an obstacle. As discussed herein, expression of MMPs in diabetic wounds is altered and contributes to the refractory nature of the wounds to heal.

MMPs are expressed as inactive zymogens (proMMPs), requiring proteolytic removal of the pro domain for their activation, which is mediated by other proteinases, including MMPs. MMPs are further regulated by complexation with tissue inhibitors of matrix metalloproteinases (TIMPs), which block access to the active site. Furthermore, MMPs are expressed at low levels in healthy tissues, but their expression increases during many diseases that involve remodeling of the ECM. This is known to be the case for chronic wounds, except the methods that have been employed do not differentiate among proMMPs and TIMP-complexed MMPs (both inactive as enzymes) and activated MMPs. It is the active MMPs that play roles in the physiology of wound healing and in the pathology of wounds refractory to healing.

Accordingly, there is a need for therapies that are effective for the treatment of chronic wounds. There is also a need for selective MMP inhibitors that are effective to enhance and accelerate the healing process.

### SUMMARY

Selective matrix metalloproteinase (MMP) inhibitors have been found to facilitate healing of diabetic wounds. It has been discovered that a number of selective inhibitor compounds significantly accelerate the healing process of various chronic wounds. The evaluations described herein demonstrate that these compounds are indeed efficacious in accelerating the healing process in diabetic mammals. Notably, the therapy was effective in diabetic mice but not in non-diabetic mice. The non-diabetic mice treated with an MMP inhibitor failed to show any acceleration effect for their wound healing. These compounds are the first discovered for this type of therapy. There are no current clinical agents that can accelerate the wound healing process in diabetics, therefore the compounds, compositions, and methods described herein will be of significant importance to patients and practitioners in need of therapeutic methods for treating chronic wounds.

The invention thus provides methods of accelerating the healing process of a skin wound. The methods can include administering to a mammal afflicted with a skin wound an effective amount of an MMP inhibitor, or a pharmaceutically acceptable salt thereof, wherein the gelatinase inhibitor accelerates the healing process of the skin wound.

The invention also provides methods of inhibiting the progression of a skin wound associated disease state characterized by elevated levels of matrix metalloproteinases. The methods can include administering to a mammal afflicted with a skin wound an effective amount of a gelatinase inhibitor, or a pharmaceutically acceptable salt thereof, effective to inhibit the progression of the skin wound in the mammal.

The invention further provides a method for enhancing the rate of repair of a diabetic skin wound. The method can include administering to the skin wound an effective amount of a gelatinase inhibitor, or a pharmaceutically acceptable salt thereof, wherein the rate of repair of the skin wound is enhanced, for example, compared to the rate of repair of a skin wound not receiving administration of the gelatinase inhibitor.

The invention additionally provides a dressing or patch for a chronic skin wound. The dressing or patch can include an effective amount of a gelatinase inhibitor, or a pharmaceutically acceptable salt thereof, and a pharmaceutically acceptable carrier, diluent, or excipient. For example, the active can be included in an ointment base, where the gelatinase inhibitor and the ointment base are combined and incorporated into a dressing. The dressing can a woven or non-woven fabric and can further include a backing and/or an adhesive.

The MMP inhibitor can be any suitable and effective gelatinase inhibitor or collagenase inhibitor. Examples of various gelatinase inhibitors and collagenase inhibitors are described, recited, illustrated, or referenced herein. The suitable compounds can include their salts, solvates, or prodrugs. Examples of effective inhibitors include SB-3CT, p-amino SB-3CT, p-hydroxy SB-3CT, and p-Arg SB-3CT.

In some embodiments, the effective amount of the gelatinase inhibitor can be, for example, about 0.01 to about 50 mg per day, about 0.1 to about 10 mg per day, about 0.5 to about 5 mg per day, or about 0.5 to about 2.5 mg per day. The effective amount of the gelatinase inhibitor can be applied, for example, topically, optionally in combination with other actives and/or carriers. The amount per day can be an amount in a composition applied, for example, topically or transdermally, or it can be an amount administered by another means, such as subdermally. For topical administration, the amount can also be about 0.01 to about 50 mg per day, about 0.1 to about 10 mg per day, about 0.5 to about 5 mg per day, or about 0.5 to about 2.5 mg per 100 cm² of wound on the surface of the patient being treated.

In some embodiments, the skin wound is a chronic skin wound. Subjects having wounds treatable by the methods described herein include mammals, such as humans. In some cases, the mammal can be suffering from diabetes, and the skin wound can be a chronic diabetic skin wound. The inhibitor can be delivered to the skin wound in a variety of forms, such as in an ointment, or the administration of the inhibitor can be intraperitoneal, such as intravenous administration.

The invention therefore provides therapeutic methods of treating skin wounds in a mammal. The methods can include administering to a mammal having a wound, such as a chronic skin wound, an effective amount of a compound or composition described herein. Mammals include primates, humans, rodents, canines, felines, bovines, ovines, equines, swine, caprines and the like.

The invention also provides compounds useful for treating wounds of the integument (e.g., skin ulcers and any break or damage to the integument) or wounds as a result of surgery, which can include systemic treatment to aid the healing of such internal wounds.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A is a graph depicting the wound closure (percentage of original wound diameter) per day in female diabetic mice in Example 1. Wound closure was determined every day using the initial and final wound diameters, and the percentage wound closure calculated as [(initial−final)/initial]×100.

FIG. 1B is a graph depicting the wound closure (percentage of original wound diameter) per day in wild-type mice (top) and diabetic mice (bottom); the gelatinase inhibitor was p-amino SB-3CT.

FIG. 2A is a graph depicting the wound closure (percentage of original wound diameter) per day in female diabetic mice in Example 3. Wound closure was determined every day using the initial and final wound diameters, and the percentage wound closure calculated as [(initial−final)/initial]×100.

FIG. 2B is a photograph of the skin lesion of a representative mouse treated with p-amino SB-3CT at 0.25 mg/wound on day 13 of Example 3.

FIG. 2C is a photograph of the skin lesion of a representative mouse treated with saline (vehicle) at 50 μL/wound on day 13 of Example 3.

FIG. 2D depicts four images from in situ gelatin zymography of the wound tissue of diabetic mice after treatment with p-amino SB-3CT at 0.25 mg/wound (top images) and saline (vehicle) at 50 μL/wound (bottom images) on day 13 of Example 3. The wound tissue of mice treated with vehicle showed gelatinolytic activity (bottom images). Wound gelatinolytic activity is visualized using fluorescein isothiocyanate (FITC)-labeled substrate (right images), and 4′,6-diamidino-2-phenylindole (DAPI)-labeled substrate (left images). The wound tissue of mice treated with p-amino SB-3CT at 0.25 mg/wound showed significantly less gelatinolytic activity (top images) than the wounds of the mice treated with vehicle (bottom images).

FIGS. 3-16 illustrate various MMP inhibitors that can be used in the methods described herein for treating wounds, according to various embodiments. However, some of the MMP inhibitors are broad-spectrum inhibitors, therefore if they inhibit MMP-8 and/or they do not inhibit MMP-9, they will not be suitable inhibitors for use with the techniques described herein.

FIG. 3. Collagen-based peptidomimetic hydroxamates.

FIG. 4. Peptidomimetic hydroxamates and carboxylates.

FIG. 5. Diaryl ether hydroxamates.

FIG. 6. Peptidomimetic hydroxamates.

FIG. 7. Peptidomimetic carboxylates.

FIG. 8. Thiol-based MMP inhibitors.

FIG. 9. Pyrimidine-based MMP inhibitors.

FIG. 10. A pyrone MMP inhibitor.

FIG. 11. Phosphine MMP inhibitors.

FIG. 12. N-Sulfonyl aminophosphonate MMP Inhibitors.

FIG. 13. A bisphosphonate MMP Inhibitor.

FIG. 14. A chemically modified tetracycline MMP inhibitor.

FIG. 15. Various competitive MMP inhibitors.

FIG. 16. Phosphorus-based MMP Inhibitors.

FIG. 17. Various mechanism-based MMP Inhibitors, including inhibitors selective for MMP-9.

FIG. 18. Active MMP-8, MMP-9, and MMP-14 are found in diabetic wounds.

FIG. 19. Identification and quantification of active MMPs in the course of diabetic wound-healing. Female diabetic (db/db) and wild-type mice received a single excisional 8-mm diameter wound in the dorsal region and were treated with 50 μL of saline once a day starting one day after wound infliction. (a) Broad-spectrum MMP inhibitor-tethered resin (compound 1). (b) Gelatin zymography of wound tissue extracts from db/db mice; proMMP-2, proMMP-9, and active MMP-2 are observed, however active MMP-9 is not detectable. The faint band below MMP-9 dimer on days 7, 10, and 14 might be the previously reported complex between MMP-8 and MMP-9. (c) Levels of active MMP-8 and MMP-9 in wound tissues quantified by the inhibitor-tethered resin coupled with nano UPLC with MRM detection. Data represent mean±SD, n=3; *p<0.05, #p<0.01. The increases in levels of active MMP-8 on day 10 were statistically significant in both wild-type and diabetic wounds, whereas active MMP-9 was upregulated only in diabetic wounds. These data indicate a detrimental role for MMP-9 and a possible beneficial role for MMP-8 in diabetic wound repair. Because active MMP-2 is not detected with the resin, the active MMP-2 band seen by gelatin zymography represents TIMP-inhibited MMP-2, a non-covalent complex. The SDS used in gelatin zymography denatures the TIMP-MMP complex, exposing the active site. Thus, detection of TIMP-inhibited gelatinases is a major drawback of gelatin zymography.

FIG. 20. ND-322 accelerates wound healing by re-epithelialization and abrogates MMP-9 activity in db/db wounds. Female db/db and wild-type mice received a single excisional 8-mm diameter wound in the dorsal region and were treated with ND-322 (50 μL of 5.0 mg/mL ND-322 in saline, equivalent to 0.25 mg/wound/day) or saline.

FIG. 20(a) Chemical structure of inhibitor 2 (also known as ND-322).

FIG. 20(b) Wound closure in db/db and wild-type mice as determined by taking photographs at the indicated time points. Wound area was calculated at each time point using photographs taken at a fixed distance above the wound and ImageJ software and expressed as percentage of wound area relative to that at day 0. Data given as mean±SD; n=35 on day 1, n=28 on day 3, n=21 on day 7, n=14 on day 10, and n=7 on day 14; *p<0.05, *p<0.01 indicate statistically significant differences in wound closure between ND-322-treated and vehicle-treated db/db mice. Differences in average wound closures in ND-322-treated and vehicle-treated wild-type mice are not statistically significant (p>0.25) on days 1, 3, 7, 10, 14. Enlargement of wounds on day 1 are due to wound retraction.

FIG. 20(c) and (d). Representative wound images in (c) wild-type and (d) db/db mice. A photo of the wound in each panel is given to the left (all to the same scale) and H&E staining to the right for day 14 after wound infliction. Wounds of vehicle-treated wild-type, ND-322-treated wild-type, and ND-322-treated db/db mice were completely re-epithelialized (indicated by dotted line) with hair growth, while those of vehicle-treated db/db mice showed partial re-epithelialization (dotted line) with no hair growth. Scale bars in panels c and d are 100 μm.

FIG. 20(e). In situ gelatin zymography with MMP fluorogenic substrate DQ-gel (green in left panels) merged with nuclear DNA staining by DAPI (blue). The extracellular MMP-9 activity (green) surrounds the nucleus. ND-322 significantly reduced gelatinolytic activity in db/db wounds compared to vehicle-treated control. Scale bars, 25 μm.

### DETAILED DESCRIPTION

Chronic wounds affect millions of individuals in the US every year. Chronic wounds include diabetic foot ulcers, pressure ulcers, and venous ulcers. These wounds do not follow a normal, predictable course of healing and can take an extended time to heal. Ischemia is an important factor contributing to the formation and persistence of wounds, causing tissue inflammation and releasing chemokines, leukotrienes, and complement factors that recruit leukocytes. Leukocytes migrate into tissue, where they express proinflammatory cytokines and produce reactive oxygen species (ROS). ROS damages cells and prevent wound closure by damaging the extracellular matrix (ECM) and cytokines that accelerate healing.

The ECM is a complex network of proteins and proteoglycans that surrounds cells and provides physical support of cells in tissue. Collagen is a major component of the ECM. A family of 26 zinc-dependent endopeptidases are responsible for the turnover and degradation of the ECM, including its collagen. These endopeptidases are also known as matrix metalloproteinases (MMPs). Gelatinases A (MMP-2) and B (MMP-9) are able to break down collagen more effectively than other MMPs. They also cleave collagen type IV, the major constituent of the basement membrane. In addition, MMP-2 has been shown to play an important role in the reorganization of collagen lattices. These enzymes are inhibited by tissue inhibitors of MMP (TIMP) and are believed to be responsible for the increased destruction of the ECM observed in chronic wounds. Fluids from chronic human wounds have elevated levels of pro-inflammatory cytokines, including tumor necrosis factor-alpha and interleukin-1b, and elevated levels of MMPs and serine proteases.

Marked upregulation of MMP-2 and MMP-9 is found in chronic wounds. Higher levels of MMP-9 in chronic wound fluid correlate with clinically more severe wounds. Reduced levels of TIMP are also found in chronic wounds. As described herein, it has now been determined that selective gelatinase inhibitors can be effective in the treatment of chronic wounds.

The discovery and synthesis of 2-(((4-phenoxyphenyl)sulfonyl)methyl)thiirane (SB-3CT; compound (1)), the first prototype mechanism-based inhibitor for MMPs (Kᵢ 14±1 nM and 600±200 nM for human MMP-2 and MMP-9, respectively), was reported in 2000 (Brown et al., J. Am. Chem. Soc. 2000, 122, 6799-6800; Toth et al., J. Biol. Chem. 2000, 275, 41415-23). SB-3CT has been found to be effective in animal models of prostate cancer metastasis to the bone, breast cancer metastasis to the lungs, T-cell lymphoma metastasis to the liver, ischemic stroke, subarachnoid hemorrhage, spinal cord injury, traumatic brain injury, and testosterone-induced neurogenesis.

### DEFINITIONS

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001; Mosby's Medical Dictionary, 8^{th} Edition, 2009, Elsevier; and The American Heritage Medical Dictionary, 2007, Houghton Mifflin Company.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is disubstituted.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

Specific values listed below for radicals, substituents, and ranges, are for illustration only; they do not exclude other defined values or other values within defined ranges for the radicals and substituents.

The term “alkyl” refers to a branched, unbranched, or cyclic hydrocarbon having, for example, from 1-20 carbon atoms, and often 1-12, 1-10, 1-8, 1-6, or 1-4 carbon atoms. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below. The alkyl can also be optionally partially or fully unsaturated. As such, the recitation of an alkyl group includes both alkenyl and alkynyl groups. The alkyl can be a monovalent hydrocarbon radical, as described and exemplified above, or it can be a divalent hydrocarbon radical (i.e., an alkylene).

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted, as described for alkyl groups.

The term “heteroaryl” refers to a monocyclic, bicyclic, or tricyclic ring system containing one, two, or three aromatic rings and containing at least one nitrogen, oxygen, or sulfur atom in an aromatic ring, and that can be unsubstituted or substituted, for example, with one or more, and in particular one to three, substituents, as described in the definition of “substituted”. Typical heteroaryl groups contain 2-20 carbon atoms in addition to the one or more hetoeroatoms. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or —(C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

The term “heterocycle” refers to a saturated or partially unsaturated ring system, containing at least one heteroatom selected from the group oxygen, nitrogen, silicon, and sulfur, and optionally substituted with one or more groups as defined for the term “substituted”. A heterocycle can be a monocyclic, bicyclic, or tricyclic group. A heterocycle group also can contain an oxo group (═O) or a thioxo (═S) group attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholinyl, piperazinyl, piperidinyl, pyrazolidinyl, pyrazolinyl, pyrrolidine, pyrroline, quinuclidine, tetrahydrofuranyl, and thiomorpholine.

The term “substituted” indicates that one or more hydrogen atoms on the group indicated in the expression using “substituted” is replaced with a “substituent”, such as for a substituted alkyl, aryl, or amino group. The number referred to by ‘one or more’ can be apparent from the moiety one which the substituents reside. For example, one or more can refer to, e.g., 1, 2, 3, 4, 5, or 6; in some embodiments 1, 2, or 3; and in other embodiments 1 or 2. The substituent can be one of a selection of indicated groups, or it can be a suitable group known to those of skill in the art, provided that the substituted atom's normal valency is not exceeded, and that the substitution results in a stable compound. Suitable substituent groups include, e.g., alkyl, alkenyl, alkynyl, alkoxy, halo, haloalkyl, hydroxy, hydroxyalkyl, aryl, aroyl, (aryl)alkyl (e.g., benzyl or phenylethyl), heteroaryl, heterocycle, cycloalkyl, alkanoyl, alkoxycarbonyl, amino, alkylamino, dialkylamino, trifluoromethyl, trifluoromethoxy, trifluoromethylthio, difluoromethyl, acylamino, nitro, carboxy, carboxyalkyl, keto, thioxo, alkylthio, alkylsulfinyl, alkylsulfonyl, arylsulfinyl, arylsulfonyl, heteroarylsulfinyl, heteroarylsulfonyl, heterocyclesulfinyl, heterocyclesulfonyl, phosphate, sulfate, hydroxylamine, hydroxyl (alkyl)amine, and cyano. Additionally, suitable substituent groups can be, e.g., —X, —R, —O⁻, —OR, —SR, —S⁻, —NR₂, —NR₃, ═NR, —CX₃, —CN, —OCN, —SCN, —N═C═O, —NCS, —NO, —NO₂, ═N₂, —N₃, —NC(═O)R, —C(═O)R, —C(═O)NRR, —S(═O)₂O⁻, —S(═O)₂OH, —S(═O)₂R, —OS(═O)₂OR, —S(═O)₂NR, —S(═O)R, —OP(═O)O₂RR, —P(═O)O₂RR, —P(═O)(O⁻)₂, —P(═O)(OH)₂, —C(═O)R, —C(═O)X, —C(S)R, —C(O)OR, —C(O)O⁻, —C(S)OR, —C(O)SR, —C(S)SR, —C(O)NRR, —C(S)NRR, or —C(NR)NRR, where each X is independently a halogen (“halo”): F, Cl, Br, or I; and each R is independently H, alkyl, aryl, (aryl)alkyl (e.g., benzyl), heteroaryl, (heteroaryl)alkyl, heterocycle, heterocycle(alkyl), or a protecting group. As would be readily understood by one skilled in the art, when a substituent is keto (═O) or thioxo (═S), or the like, then two hydrogen atoms on the substituted atom are replaced. In some embodiments, one or more of the substituents above are excluded from the group of potential values for substituents on the substituted group.

The term “interrupted” indicates that another group is inserted between two adjacent carbon atoms (and the hydrogen atoms to which they are attached (e.g., methyl (CH₃), methylene (CH₂) or methine (CH))) of a particular carbon chain being referred to in the expression using the term “interrupted, provided that each of the indicated atoms' normal valency is not exceeded, and that the interruption results in a stable compound. Suitable groups that can interrupt a carbon chain include, e.g., with one or more non-peroxide oxy (—O—), thio (—S—), imino (—N(H)—), methylene dioxy (—OCH₂O—), carbonyl (—C(═O)—), carboxy (—C(═O)O—), carbonyldioxy (—OC(═O)O—), carboxylato (—OC(═O)—), imine (C═NH), sulfinyl (SO) and sulfonyl (SO₂). Alkyl groups can be interrupted by one or more (e.g., 1, 2, 3, 4, 5, or about 6) of the aforementioned suitable groups. The site of interruption can also be between a carbon atom of an alkyl group and a carbon atom to which the alkyl group is attached.

Selected substituents within the compounds described herein may be present to a recursive degree. In this context, “recursive substituent” means that a substituent may recite another instance of itself. Because of the recursive nature of such substituents, theoretically, a large number may be present in any given claim. One of ordinary skill in the art of medicinal chemistry and organic chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis. In some embodiments, the substitution will result in a compound having a molecular weight of less than about 1200 Da, less than about 1000 Da, less than about 900 Da, less than about 800 Da, less than about 750 Da, less than about 700 Da, less than about 650 Da, less than about 600 Da, less than about 500 Da, or less than about 400 Da.

Recursive substituents are an intended aspect of the invention. One of ordinary skill in the art of medicinal and organic chemistry understands the versatility of such substituents. To the degree that recursive substituents are present in an embodiment, the total number will be determined as set forth above.

The term “amino acid” refers to a natural amino acid residue (e.g. Ala, Arg, Asn, Asp, Cys, Glu, Gln, Gly, His, Hyl, Hyp, Be, Leu, Lys, Met, Phe, Pro, Ser, Thr, Trp, Tyr, and Val) in D or L form, as well as unnatural amino acid (e.g. phosphoserine; phosphothreonine; phosphotyrosine; hydroxyproline; gamma-carboxyglutamate; hippuric acid; octahydroindole-2-carboxylic acid; statine; 1,2,3,4-tetrahydroisoquinoline-3-carboxylic acid; penicillamine; ornithine; citruline; a-methyl-alanine; para-benzoylphenylalanine; phenylglycine; propargylglycine; sarcosine; and tert-butylglycine) residue having one or more open valences. The term also comprises natural and unnatural amino acids bearing amino protecting groups (e.g. acetyl, acyl, trifluoroacetyl, or benzyloxycarbonyl), as well as natural and unnatural amino acids protected at carboxy with protecting groups (e.g. as a (C₁-C₆)alkyl, phenyl or benzyl ester or amide). Other suitable amino and carboxy protecting groups are known to those skilled in the art (see for example, T. W. Greene, Protecting Groups In Organic Synthesis; Wiley: New York, Third Edition, 1999, and references cited therein; D. Voet, Biochemistry, Wiley: New York, 1990; L. Stryer, Biochemistry, (3rd Ed.), W.H. Freeman and Co.: New York, 1975; J. March, Advanced Organic Chemistry, Reactions, Mechanisms and Structure, (2nd Ed.), McGraw Hill: New York, 1977; F. Carey and R. Sundberg, Advanced Organic Chemistry, Part B: Reactions and Synthesis, (2nd Ed.), Plenum: New York, 1977; and references cited therein).

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an amount effective can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” extend to prophylaxis and include prevent, prevention, preventing, lowering, stopping or reversing the progression or severity of the condition or symptoms being treated. As such, the term “treatment” includes both medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The compositions and methods described herein can be used for aiding wound management. The term “wound management” refers to therapeutic methods that induce and/or promote repair of a wound including, but not limited to, arresting tissue damage such as necrotization, promoting tissue growth and repair, reduction or elimination of an established microbial infection of the wound and prevention of new or additional microbial infection or colonization. The term can further include reducing or eliminating the sensation of pain attributable to a wound.

The therapeutic compositions for use in methods of wound management can include a surfactant that can useful in cleaning a wound or contributing to bactericidal activity of the administered compositions. Suitable surfactants include, but are not limited to, phospholipids such as lecithin, including soy lecithin and detergents. The surfactant selected for application to a wound or skin surface will typically be mild and will not lead to extensive irritation or promote further tissue damage to the patient.

Suitable nonionic surfactants that can be used include, for example, fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, and esters of sorbitol polyglycol ethers. One specific surfactant is polypropylene glycol ethoxylates, for example, with a concentration of about 5 wt % and about 25 wt %, including, for example, the poloxymer Pluronic F-127 (Poloxamer 407). In other embodiments, the surfactant can include lecithin with or without the addition of Pluronic F-127, the Pluronic F-127 being about 2 and about 20 wt % for increasing the viscosity or gelling of the compositions.

A “wound” refers to an injury to the body, including but not limited to an injury from trauma, violence, accident, or surgery. A wound may occur due to laceration or breaking of a membrane (such as the skin) and usually damage to underlying tissues. A wound may occur in a topical location or internally. Chronic wounds may be caused by diseases, including but not limited to diabetes; diseases of internal organs, including but not limited to diseases of the liver, kidneys or lungs; cancer; or any other condition that slows the healing process.

Natural healing occurs in clearly defined stages. Skin wounds of acute nature may heal in 1-3 weeks in a biological process that restores the integrity and function of the skin and the underlying tissue. Such wounds may be the result of a scrape, abrasion, cut, graze, incision, tear, or bruise to the skin. If a wound does not heal in 4-12 weeks, it may be considered chronic. In the chase of chronic wounds, the wound may be attenuated at one of the stages of healing or fail to progress through the normal stages of healing. A chronic wound may have been present for a brief period of time, such as a month, or it may have been present for several years.

The phrase “chronic skin wound” includes, but is not limited to, skin ulcers, bed sores, pressure sores, diabetic ulcers and sores, and other skin disorders. Chronic skin wounds can be any size, shape or depth, and may appear discolored as compared to normal, healthy skin pigment. Chronic skin wounds can bleed, swell, seep pus or purulent discharge or other fluid, cause pain or cause movement of the affected area to be difficult or painful. Chronic skin wounds can become infected, producing elevated body temperatures, as well as pus or discharge that is milky, yellow, green, or brown in color, and is odorless or has a pungent odor. If infected, chronic skin wounds may be red, tender, or warm to the touch.

Chronic skin wounds can be caused by diabetes, poor blood supply, low blood oxygen, by conditions where blood flow is decreased due to low blood pressure, or by conditions characterized by occluded, blocked or narrowed blood vessels. A low oxygen supply can be caused by certain blood, heart, and lung diseases, and/or by smoking cigarettes. Chronic skin wounds can also be the result of repeated trauma to the skin, such as swelling or increased pressure in the tissues, or constant pressure on the wound area. Chronic skin wounds can be caused by a weakened or compromised immune system. A weakened or compromised immune system can be caused by increasing age, radiation, poor nutrition, and/or medications, such as anti-cancer medicines or steroids. Chronic skin wounds can also be cause by bacterial, viral or fungal infections, or the presence of foreign objects.

The term “diabetes” refers to any of several metabolic conditions characterized by the excessive excretion of urine and persistent thirst. The excess of urine can be caused by a deficiency of antidiuretic hormone, as in diabetes insipidus, or it can be the polyuria resulting from the hyperglycemia that occurs in diabetes mellitus.

The phrase “type 1 diabetes mellitus” refers to the first of the two major types of diabetes mellitus, characterized by abrupt onset of symptoms (often in early adolescence), insulinopenia, and dependence on exogenous insulin. It results from a lack of insulin production by the pancreatic beta cells. With inadequate control, hyperglycemia, protein wasting, and ketone body production occur. The hyperglycemia leads to overflow glycosuria, osmotic diuresis, hyperosmolarity, dehydration, and diabetic ketoacidosis, which can progress to nausea and vomiting, stupor, and potentially fatal hyperosmolar coma. The associated angiopathy of blood vessels (particularly microangiopathy) affects the retinas, kidneys, and arteriolar basement membranes. Polyuria, polydipsia, polyphagia, weight loss, paresthesias, blurred vision, and irritability can also occur.

The phrase “type 2 diabetes mellitus” refers to the second of the two major types of diabetes mellitus, peaking in onset between 50 and 60 years of age, characterized by gradual onset with few symptoms of metabolic disturbance (glycosuria and its consequences) and control by diet, with or without oral hypoglycemics but without exogenous insulin required. Basal insulin secretion is maintained at normal or reduced levels, but insulin release in response to a glucose load is delayed or reduced. Defective glucose receptors on the pancreatic beta cells may be involved. It is often accompanied by disease of blood vessels, particularly the large ones, leading to premature atherosclerosis with myocardial infarction or stroke syndrome.

Patients suffering from diabetes can develop chronic wounds of the skin, internal wounds from surgery, or other medical conditions that are not able to fully heal without the aid of the treatments methods described herein.

A “matrix metalloproteinase inhibitor” is a compound that inhibits one or more isoforms of an enzyme of the class of matrix metalloproteinases. Suitable and effective MMP inhibitors for the compositions and methods described herein can be collagenase inhibitors or gelatinase inhibitors. In some embodiments, the inhibitor inhibits MMP-9. In some embodiments, the inhibitor does not inhibit MMP-8. In further embodiments, the inhibitor selectively inhibits MMP-9 but not MMP-8. In various embodiments, the structure of the MMP inhibitor comprises a thiirane group, such as a methyl thiirane moiety. Examples of matrix metalloproteinase inhibitors (MMPi's) include SB-3CT (1) and substituted derivatives thereof, such as compounds 2-5.

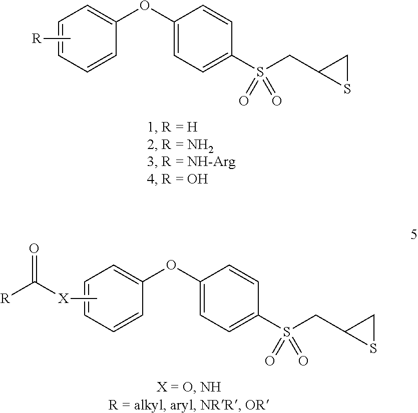

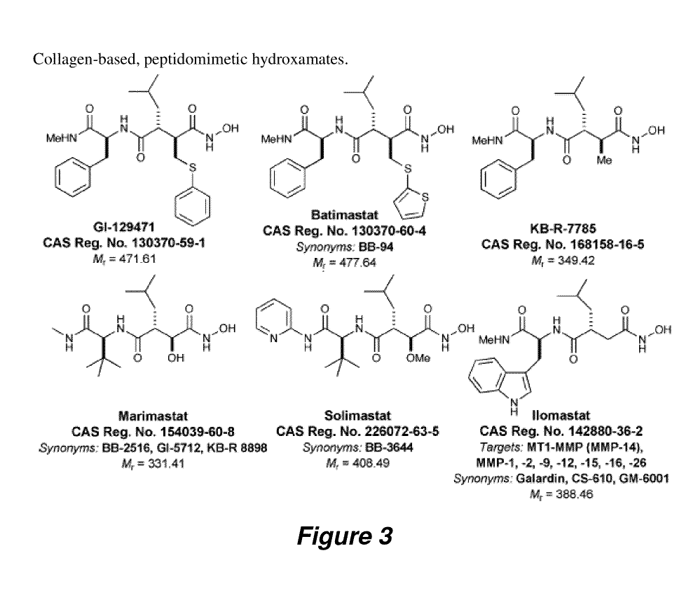

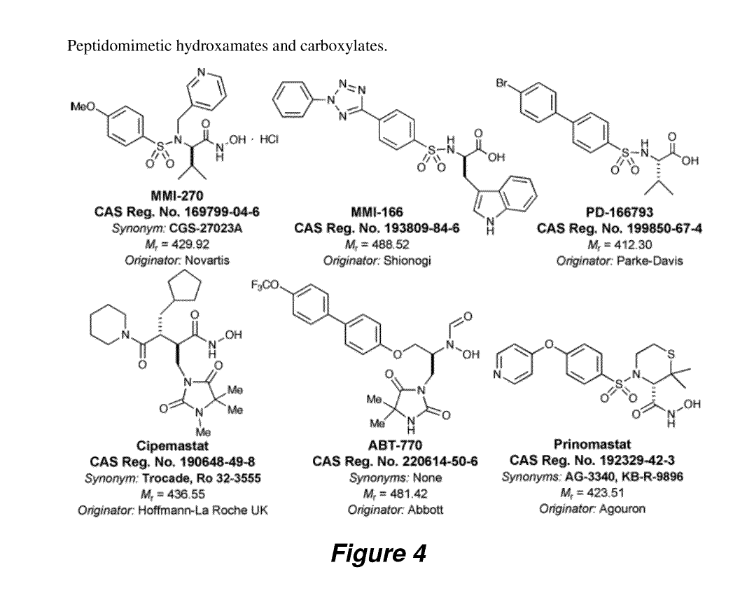

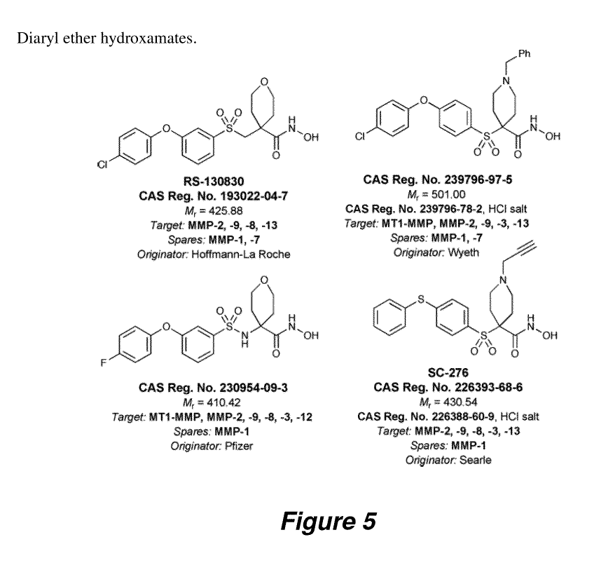

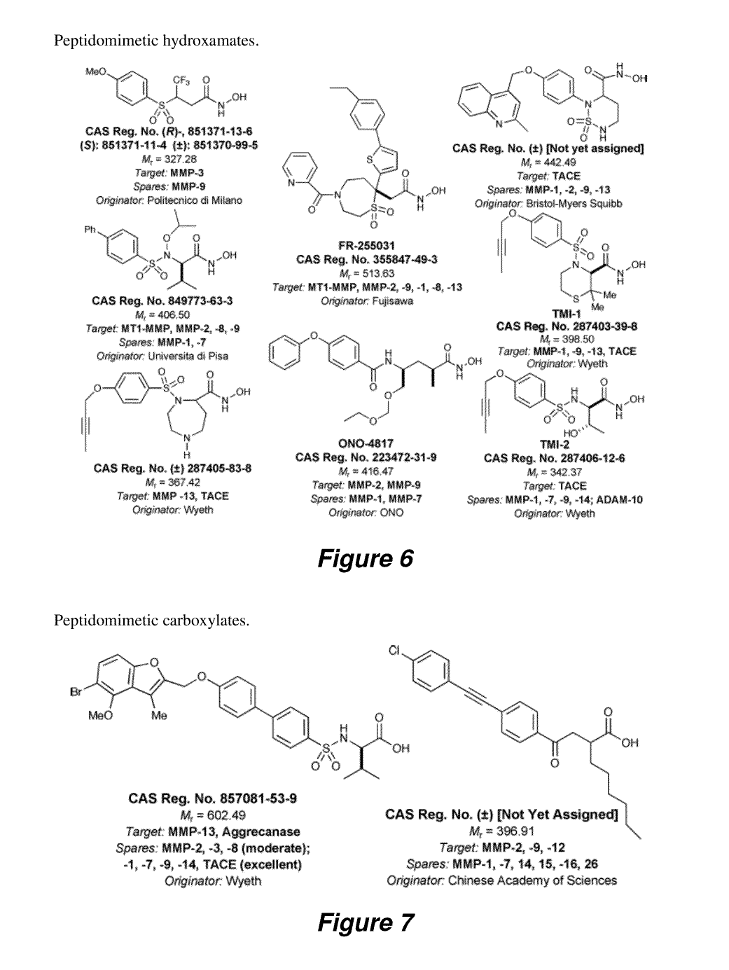

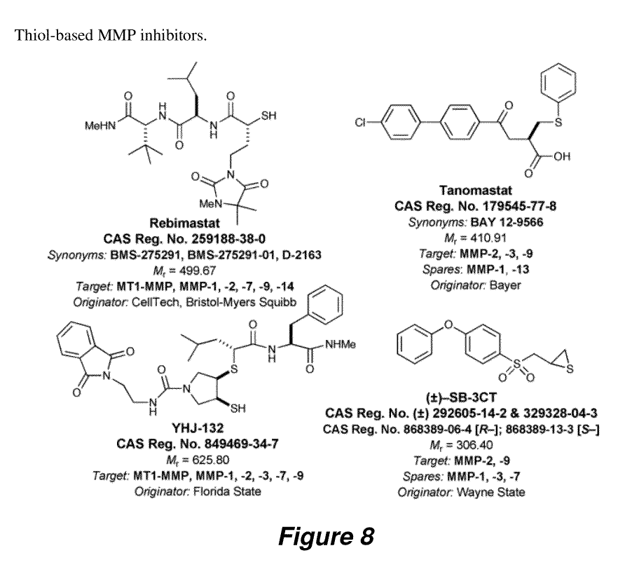

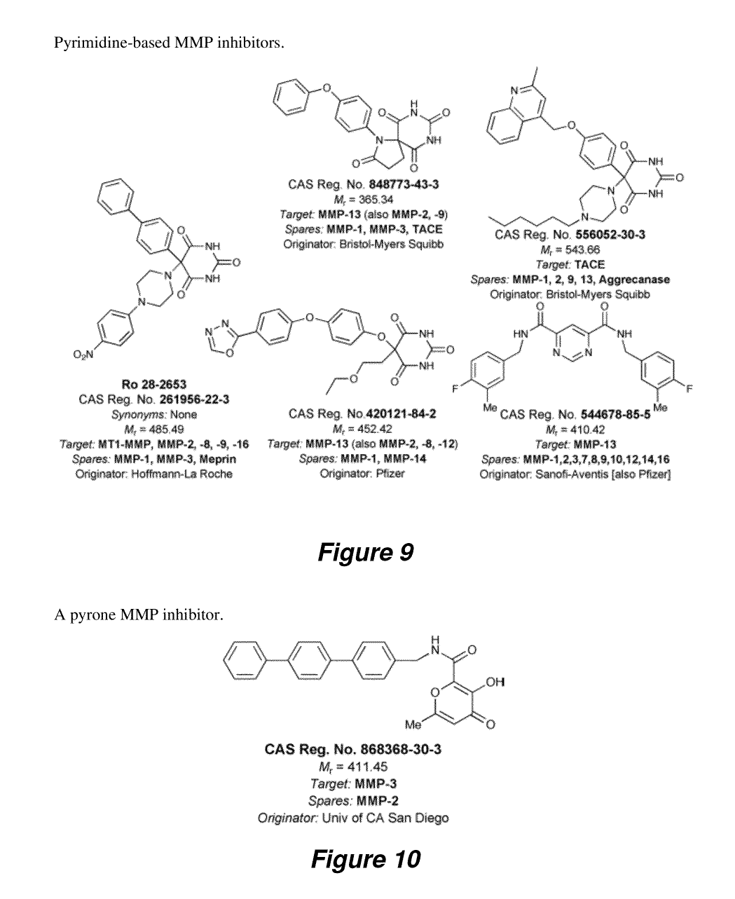

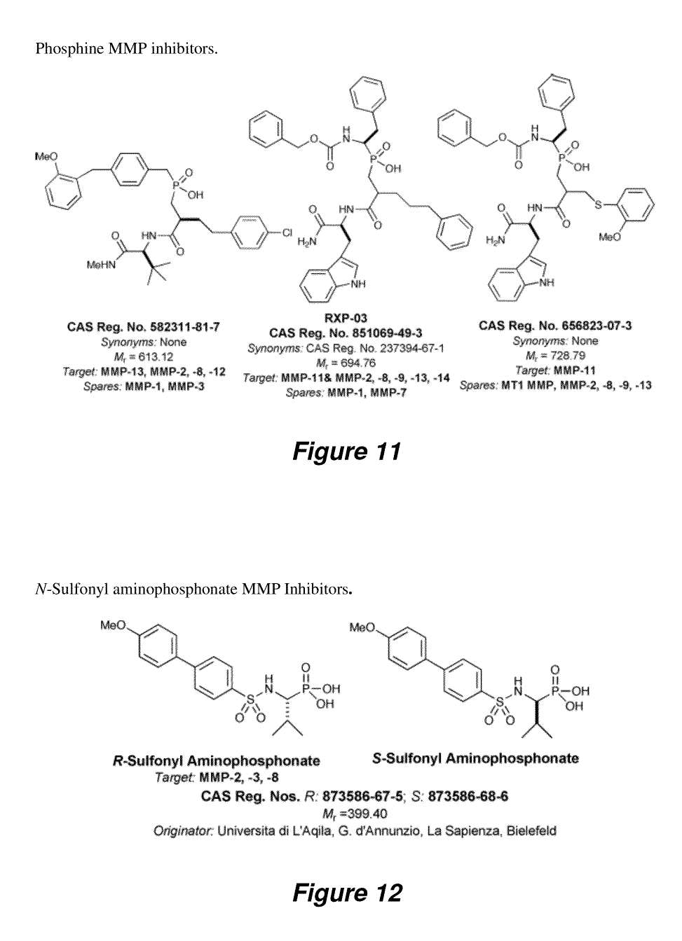

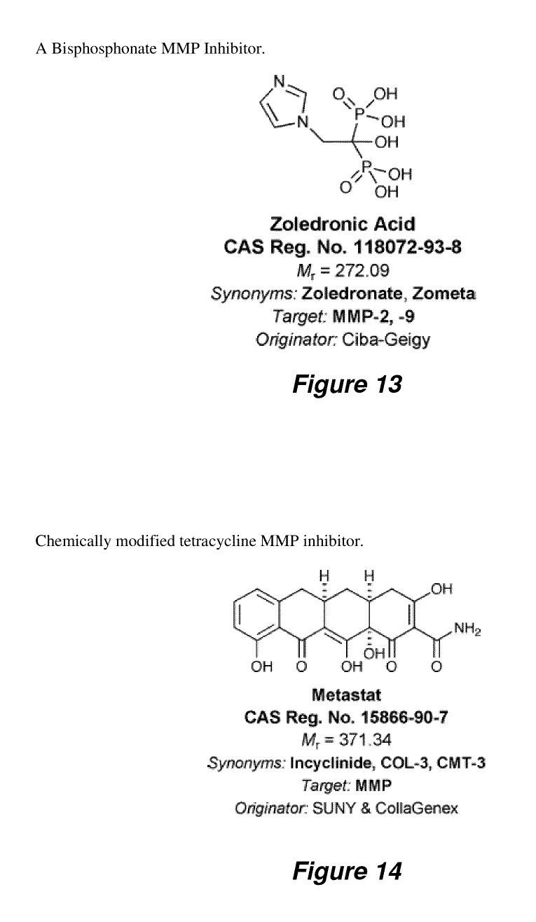

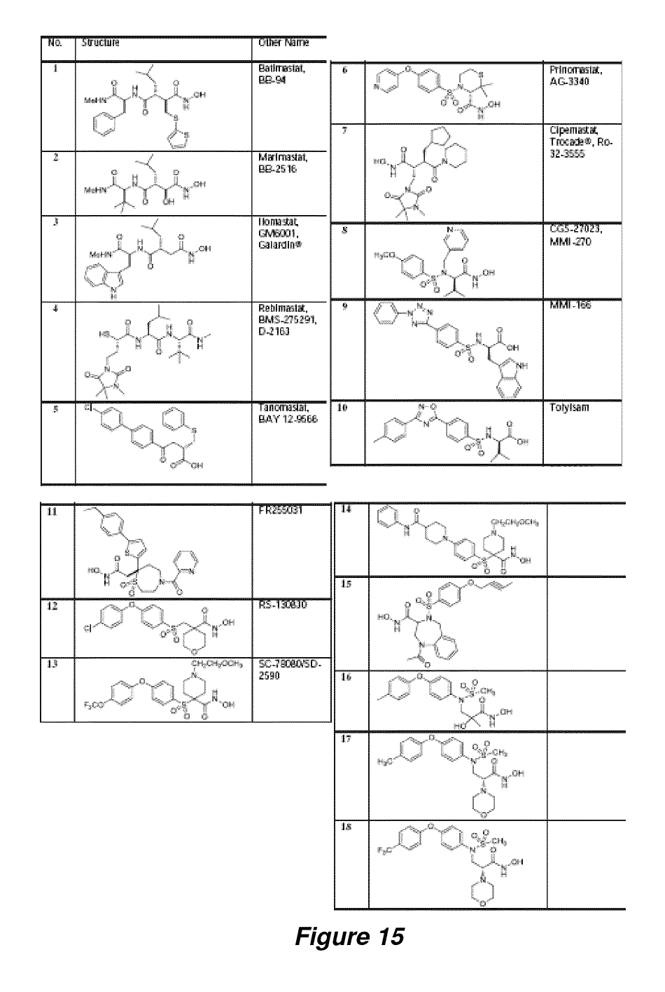

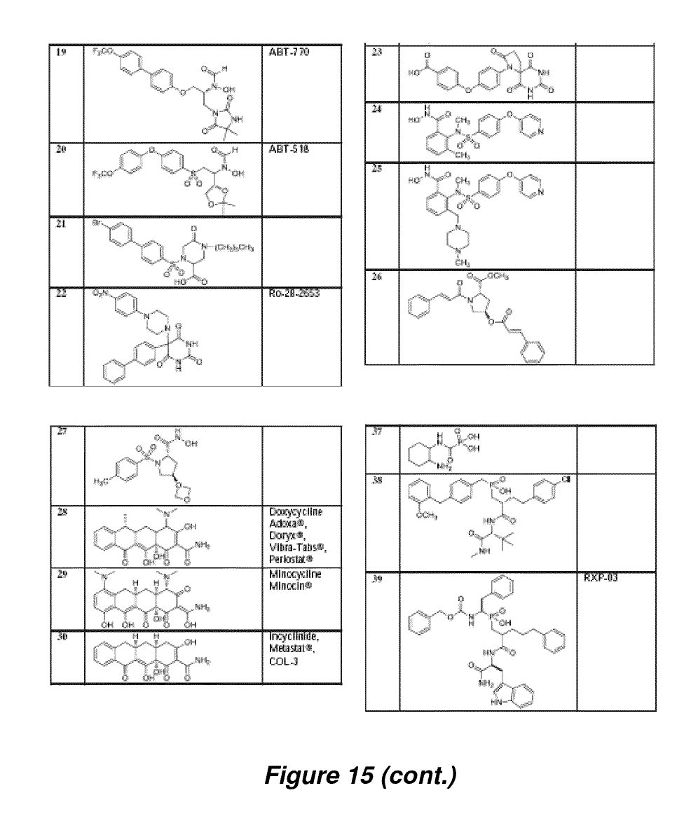

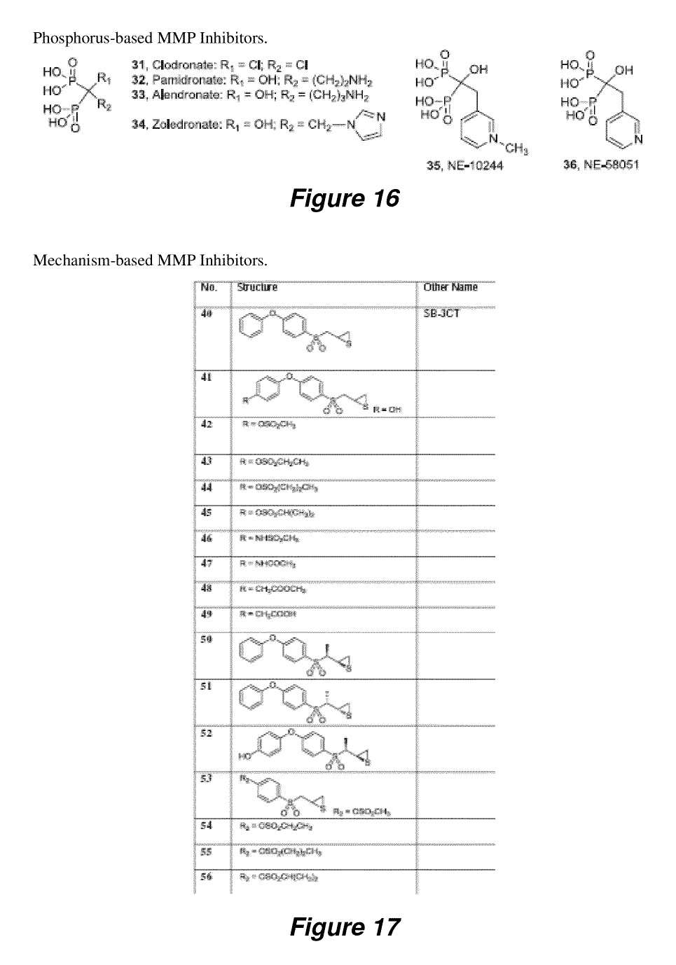

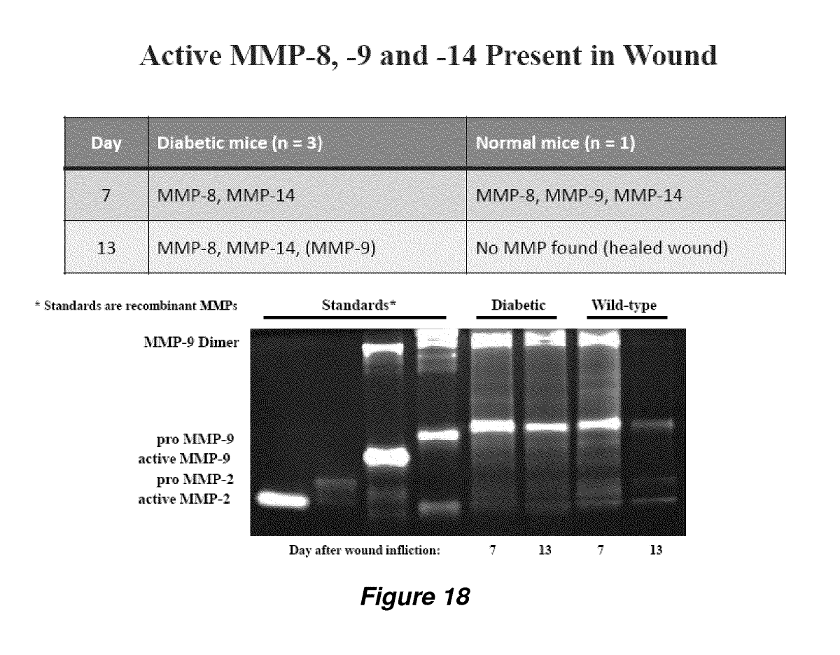

When substituted, the substituent on the phenoxy group can be ortho, meta, or para with respect to the oxygen linking the two phenyl groups. Specific examples can include SB-3CT, p-amino SB-3CT, p-hydroxy SB-3CT, or p-Arg SB-3CT. Thus, in some embodiments, the gelatinase inhibitor is a compound of Formula I:

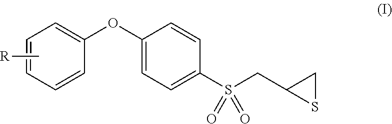

wherein R is H, OH, NH₂, NH-amino acid, or —X—(C═O)—R′ where X is O or NH, and R′ is alkyl, aryl, alkylaryl, amino, or alkoxy, where any alkyl, aryl, or amino is optionally substituted. In one specific embodiment, the amino acid can be arginine. Additional examples of suitable MMP inhibitors include the compounds disclosed in U.S. Pat. Nos. 6,703,415 (Mobashery et al.) and 7,928,127 (Lee et al.), and PCT Publication No. WO 2011/026107 (Mobashery et al.). Additional examples of MMP inhibitors are illustrated in FIGS. 3-17.

### Pharmaceutical Formulations

The compounds recited, illustrated, described, or referenced herein can be used to prepare therapeutic pharmaceutical compositions. The compounds may be added to the compositions in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, a-ketoglutarate, and a-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard or soft shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 2% to about 60% of the weight of a given unit dosage form. The amount of active compound in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally or subcutaneously by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

For topical administration, compounds may be applied in pure form, e.g., when they are liquids. However, it will generally be desirable to administer the active agent to the skin as a composition or formulation, for example, in combination with a dermatologically acceptable carrier, which may be a solid or a liquid.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Ointments are semisolid preparations that are typically based on petrolatum or other petroleum derivatives. The specific ointment base to be used, as will be appreciated by those skilled in the art, can be one that will provide for optimum active ingredients delivery and can provide for other desired characteristics such as emolliency or the like. As with other carriers or vehicles, an ointment base should be relatively inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Some water-soluble ointment bases are prepared from polyethylene glycols of varying molecular weight; again, reference may be made to Remington: The Science and Practice of Pharmacy for further information.

Examples of dermatological compositions for delivering active agents to the skin are known to the art; for example, see U.S. Pat. Nos. 4,992,478 (Geria), 4,820,508 (Wortzman), 4,608,392 (Jacquet et al.), and 4,559,157 (Smith et al.). Such dermatological compositions can be used in combinations with the compounds described herein.

Useful dosages of the compounds and compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician, practitioner, or clinician.

The compound can be conveniently administered in a unit dosage form, for example, containing 5 to 1000 m g/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The ability of a compound of the invention to treat skin wounds may be determined by using assays well known to the art. For example, the design of treatment protocols, analysis of wound tissue or fluid, toxicity evaluation, data analysis, and quantification of wound characteristics are known. In addition, the ability of a compound to treat wounds in diabetic mammals may be determined using the Tests as described below.

Medical dressings suitable for use in methods for contacting a wound with the therapeutic compositions can be any material that is biologically acceptable and suitable for placing over a chronic wound. In some embodiments, the support can be a woven or non-woven fabric of synthetic or non-synthetic fibers, or any combination thereof. The dressing can also include a support, such as a polymer foam, a natural or man-made sponge, a gel or a membrane that can absorb or have disposed thereon, a therapeutic composition. One gel suitable for use as a support for the composition is sodium carboxymethylcellulose 7H 4F.

In some embodiments, the formulation can include a permeation enhancer, such as transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), or dodecyl-2-dimethylaminopropanoate (DDAIP), which can be provided at a weight/weight concentration of about 0.1% to about 10%, usually from about 2.5% to about 7.5%, often about 5%.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Evaluation of SB-3CT in a Chronic Wound Model

Female diabetic mice (db/db mice, 9-10 weeks old, Jackson Laboratory, Bar Harbour, Me.) (n=10 per group) were prepared for aseptic surgery by clipping the hair on the dorsal thorax and scrubbing the skin with betadine followed by alcohol. Each mouse received two 6-mm skin punch biopsy lesions on the dorsal thorax, as described by Sullivan et al (Plast. Reconstr. Surg. 2004, 113, 953-960), under isoflurane anesthesia. Each wound was covered with an occlusive dressing following biopsy. The mice recovered on a cage with clean bedding on a heating pad. Mice received buprenorphine subcutaneously (1.5-2.5 mg/kg) at the conclusion of surgery and again 3-5 hours later. Mice were administered 0.5 mL of warm sterile saline intraperitoneally at the conclusion of surgery. The mice were treated topically with SB-3CT at a dose equivalent to 0.25 mg/wound, SB-3CT at a dose equivalent to 1.25 mg/wound, or vehicle (DMSO) at 50 μL/wound daily once per day for 23 days. Wound closure was determined every day for each animal using the initial and final wound diameters, with the percentage wound closure calculated as [(initial−final)/initial]×100.

On day 12, half of the mice (n=10) were sacrificed and the wounds of each animal excised. The remaining mice continued treatment at the dosages provided above once a day until day 23, when they were sacrificed On the day of sacrifice, the wounds of each mouse were excised, and analyzed by histology, gelatin zymography and Western blot, and in situ gelatin zymography. Specifically, one wound from each sacrificed animal was analyzed by in situ gelatin zymography, and one wound from each sacrificed animal was analyzed by gelatin zymography, followed by Western blot.

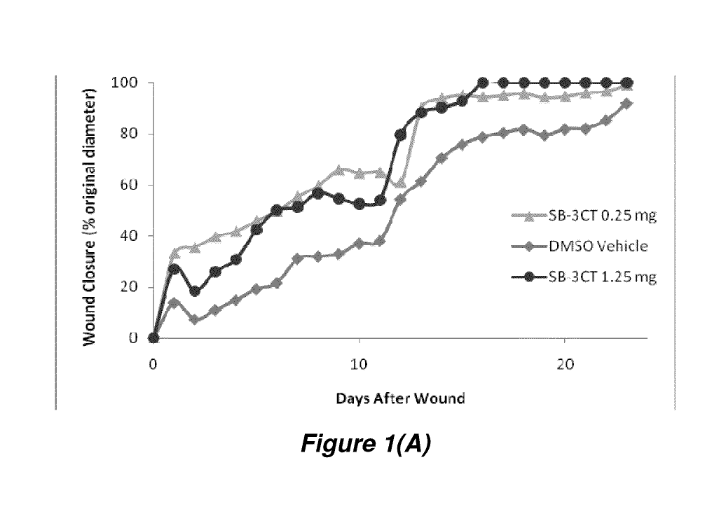

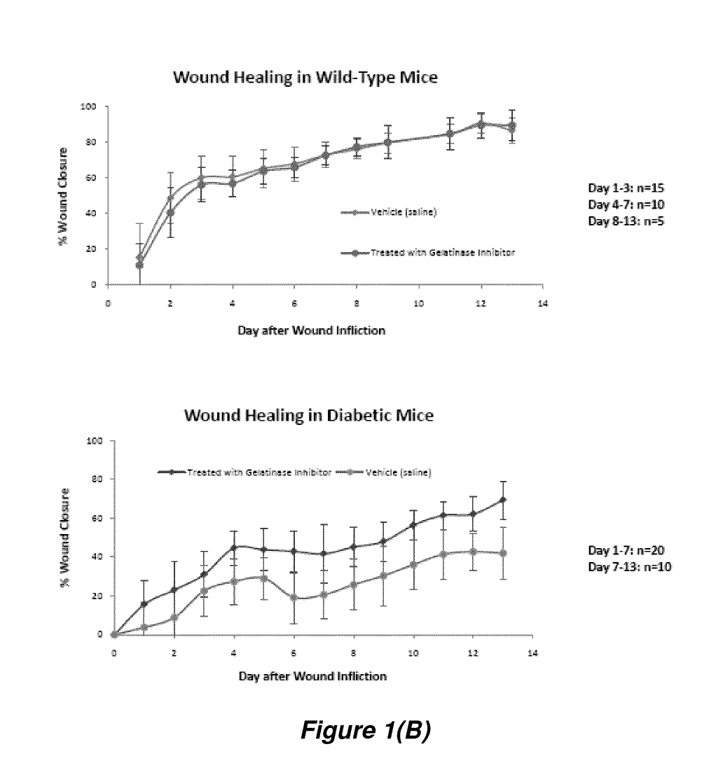

Significant differences (p<0.05) between the vehicle and SB-3CT-treated mice were observed starting on day 2 (FIG. 1). On day 6, wound closure was 49.6±14.0%, 50.0±7.4%, and 21.6±10.6% in mice receiving SB-3CT at 0.25 mg/wound per day, SB-3CT at 1.25 mg/wound per day, and vehicle (DMSO) at 50 μL/wound per day, respectively. At 13-14 days, the SB-3CT-treated mice achieved >90% wound closure. Mice given vehicle achieved >90% wound closure by day 23.

### Example 2

### Evaluation of p-Amino SB-3CT and p-Arg SB-3CT in Chronic Wound Model

Female diabetic mice (db/db mice; 9-10 weeks old, Jackson Laboratory, Bar Harbour, Me.) (n=10 per group) were prepared for aseptic surgery by clipping the hair on the dorsal thorax and scrubbing the skin with betadine followed by alcohol. Each mouse received two 6-mm skin punch biopsy lesions on the dorsal thorax, as described by Sullivan et al (Plast. Reconstr. Surg. 2004, 113, 953-960), under isoflurane anesthesia. Each wound was covered with an occlusive dressing following biopsy. The mice recovered on a cage with clean bedding on a heating pad. Mice received buprenorphine subcutaneously (1.5-2.5 mg/kg) at the conclusion of surgery and again 3-5 hours later. Mice were administered 0.5 mL of warm sterile saline intraperitoneally at the conclusion of surgery. The mice were treated topically with p-amino SB-3CT (at a dose equivalent to 0.25 mg/wound), p-Arg (at a dose equivalent to 0.25 mg/wound) or vehicle (saline) (50 μL/wound) once per day for 6 days. Wound closure was determined every day for each animal using the initial and final wound diameters, with the percentage wound closure calculated as [(initial−final)/initial]×100.

On day 6, mice treated with p-amino SB-3CT showed 37.0±9.5% wound closure; mice treated with p-Arg showed 34.7±13.2% wound closure; and mice treated with vehicle (saline) showed 21.6±10.6% wound closure. Both experimental compounds showed efficacy in this example.

### Example 3

### Evaluation of p-Amino SB-3CT in Chronic Wound Model

Female diabetic mice (db/db mice) (n=10 mice per group, n=2 wounds per mouse, 9-10 weeks old, Jackson Laboratory, Bar Harbour, Me.) were prepared for aseptic surgery by clipping the hair on the dorsal thorax and scrubbing the skin with betadine followed by alcohol. Each mouse received two 6-mm skin punch biopsy lesions on the dorsal thorax, as described by Sullivan et al (Plast. Reconstr. Surg. 2004, 113, 953-960), under isoflurane anesthesia. Each wound was covered with an occlusive dressing following biopsy. The mice recovered on a cage with clean bedding on a heating pad. Mice received buprenorphine subcutaneously (1.5-2.5 mg/kg) at the conclusion of surgery and again 3-5 hours later. Mice were administered 0.5 mL of warm sterile saline intraperitoneally at the conclusion of surgery. The mice were treated topically with p-amino SB-3CT (at a dose equivalent to 0.25 mg/wound) or vehicle (saline) at 50 μL/wound daily once per day 13 days. Wound closure was determined every day for each animal using the initial and final wound diameters, with the percentage wound closure calculated as [(initial−final)/initial]×100.

On day 7, half of the mice (n=10) were sacrificed and the wounds of each animal excised. The remaining mice continued treatment with vehicle or p-amino SB-3CT once a day until day 13, when the mice were sacrificed. On the day of sacrifice, the wounds of each mouse were excised, and analyzed by histology, gelatin zymography and Western blot, and in situ gelatin zymography. Specifically, one wound from each sacrificed animal was analyzed by in situ gelatin zymography, and one wound from each sacrificed animal was analyzed by gelatin zymography, followed by Western blot.

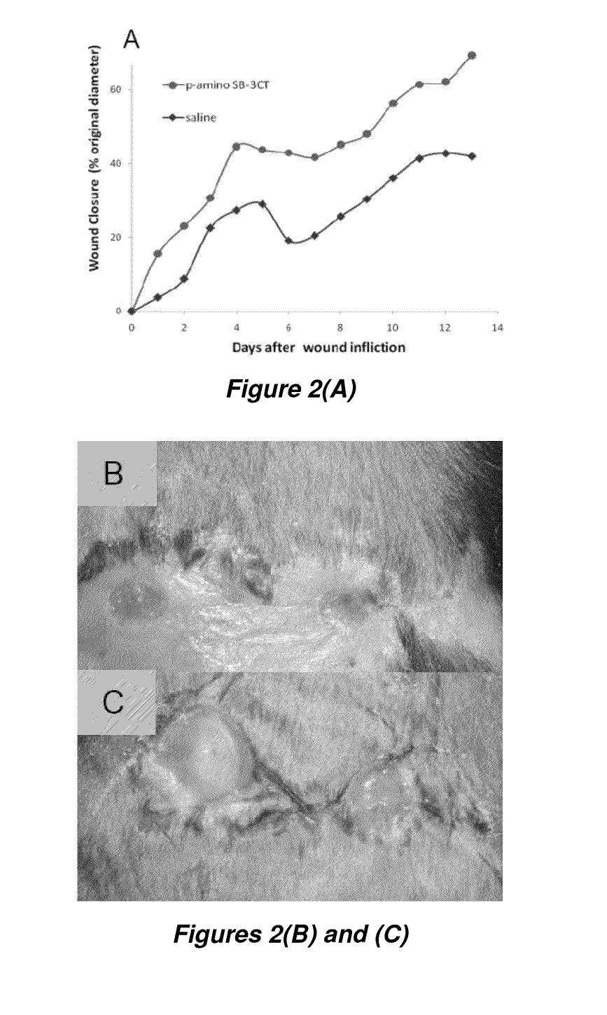

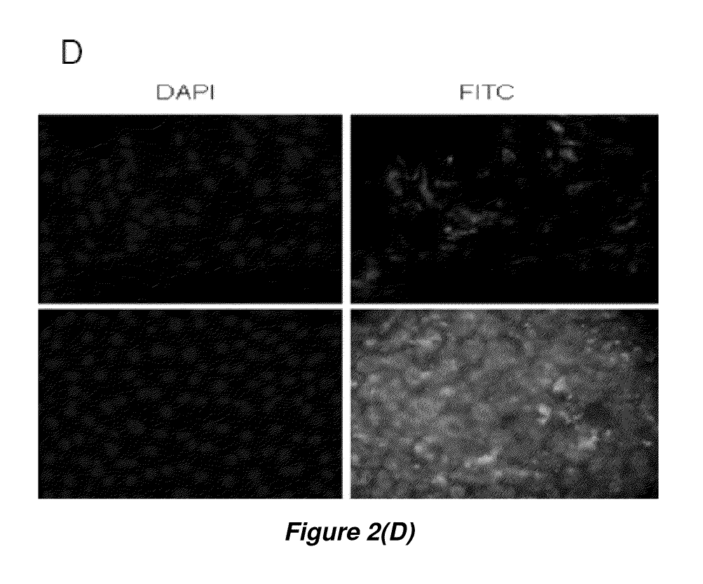

Significant differences in wound healing were observed between the treated and vehicle groups (FIGS. 2(A), 2(B), 2(C) and 2(D)). FIG. 2(A) is a graph of the wound closure measurements, determined every day of the study as [(initial−final)/initial]×100. FIG. 2(B) is a photograph of a representative skin lesion of a mouse treated with p-amino SB-3CT on day 13. FIG. 2(C) is a photograph of a representative skin lesion of a mouse treated with saline on day 13. FIG. 2(D) shows four images of in situ gelatin zymography of the wound tissue of diabetic mice after treatment with p-amino SB-3CT at 0.25 mg/wound (top images) and saline (vehicle) at 50 μL/wound (bottom images) on day 13. Wound gelatinolytic activity is visualized using fluorescein isothiocyanate (FITC)-labeled substrate (right images), and 4′,6-diamidino-2-phenylindole (DAPI)-labeled substrate (left images). The wound tissue of mice treated with vehicle showed gelatinolytic activity (bottom images). The wound tissue of mice treated with p-amino SB-3CT at 0.25 mg/wound showed suppression of gelatinolytic activity (top images), as compared to the wounds of the mice treated with vehicle (bottom images). Thus, gelatinolytic activity was suppressed in the gelatinase inhibitor-treated group (FIG. 2 (D)).

### Example 4

### Diabetic Wound Healing

Using a broad-spectrum MMP-tethered resin coupled with tandem mass spectrometry, the active MMPs present in wound tissues of diabetic mice can be identified. These studies confirm the importance of MMPs in wound healing in diabetic mice and open the doors to effective therapies for the management of wounds in diabetes.

Prodrugs 5 (Scheme 4-1), where the base compounds are derivatized with a lipophilic group, are amenable to formulation as a depot prodrug in ointment or oil. Topical treatment with a depot formulation that includes an MMP prodrug is one method to increase the duration of action of a drug. The ointment or oil formulation can serve as a drug reservoir at the site of the wounds. After application to a wound, a long duration of action occurs as a result of the slow release of the drug from the reservoir. The prodrugs 5 can slowly hydrolyze to the active gelatinase inhibitor 6 within the wound tissue to exert their efficacy.

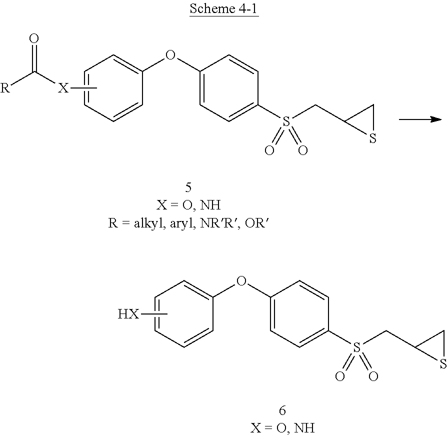

where each R and R′ group is independently H, alkyl, aryl, alkylaryl, heteroaryl, alkylheteroaryl, heterocycle, alkylheterocycle, cycloalkyl, or alkylcycloalkyl, each optionally substituted, and optionally linked to compound 5 through a tether or linking group.

The alkyl groups of Scheme 4-1 can each independently be, for example, (C₁-C₂₄)alkyl, wherein the alkyl is optionally substituted, optionally unsaturated, and optionally interrupted at carbon with one or more oxygen or nitrogen atoms, and/or one or more ester, amide, or carbamate groups.

The variable substituent on compounds 5 and 6 can be ortho, meta, or para with respect to the phenyl ether. Additional examples of suitable inhibitors include the compounds described in U.S. Pat. Nos. 6,703,415 (Mobashery et al.) and 7,928,127 (Lee et al.), and PCT Publication No. WO 2011/026107 (Mobashery et al.), each of which is incorporated herein by reference. Further examples of MMP inhibitors that can be used in therapeutic methods described herein are illustrated in FIGS. 3-17, which can also be converted into prodrugs for use in the therapeutic methods described herein.

### Example 5

### Selective Inhibition of MMP-9 Accelerates Healing of Diabetic Wounds

Chronic wounds are a complication of diabetes. With the use of a novel affinity resin that binds only the active forms of matrix metalloproteinases (MMPs), MMP-8 and MMP-9 were identified in a mouse diabetic wound model. The activity of MMP-9 makes diabetic wounds refractory to healing. Pharmacological intervention with a selective MMP-9 inhibitor led to acceleration of wound healing, accompanied by re-epithelialization.

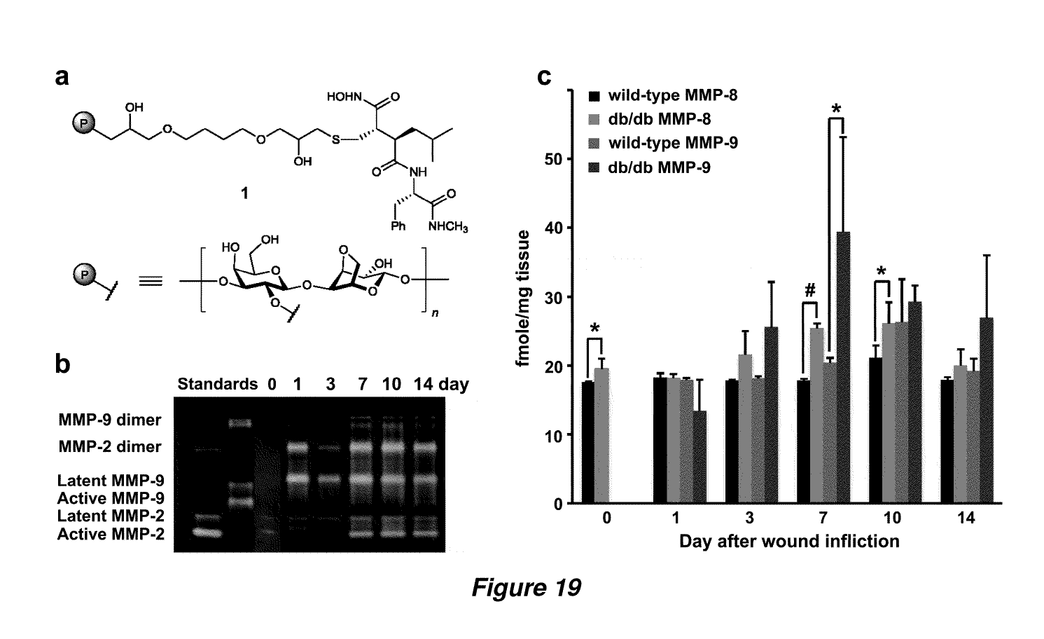

In addressing what active MMPs might play roles in disease, we have devised a resin that has been covalently tethered to a broad-spectrum MMP inhibitor (FIG. 19a, compound 1), based on the structure of batimastat. The resin binds only to active MMPs, to the exclusion of MMP zymogens and TIMP-inhibited MMPs. Bound active MMPs were detected and quantified by mass spectrometry with a limit of detection at the single-digit femtomole (10⁻¹⁵ mole) level (Hesek et al., Chem. Biol. 13 (4), 379-386 (2006)). An excision wound-healing model was used in diabetic mice, which produces wounds that undergo re-epithelialization rather than contraction as they heal, hence it is relevant to wounds in diabetic patients.

Incubation with the resin, followed by reduction of disulfide bonds in the bound proteins, alkylation to prevent disulfide linkages from recurring, digestion with trypsin, and analysis by nano ultra performance liquid chromatography (UPLC) coupled to tandem mass spectrometry identified active MMP-8 and MMP-9, and trace amounts of active MMP-14, MMP-19, ADAMS, ADAMS, ADAM 10, and ADAM 17 in wound tissues of diabetic mice (ADAMs are zinc proteases closely related to MMPs). The tissues were also analyzed by zymography, a method of choice in the field for detection of MMPs.

Zymography showed the presence of proMMP-2, active MMP-2, and proMMP-9 (FIG. 19b), whereas active MMP-9 was conspicuously not detectable by this method. A faint band of slightly lower molecular mass than the MMP-9 dimer might represent the known complex between MMP-8 and MMP-9. While MMP-2 and MMP-9 have been proposed to exist in diabetic wounds, active MMP-2 was not found in diabetic wounds with our resin. Because the resin binds only to active MMP(s), the active MMP-2 band observed by gelatin zymography (FIG. 19b) was determined to be TIMP-inhibited, resulting in an inactive form of the enzyme. In contrast, active MMP-9 is not detectable by gelatin zymography (FIG. 19b), however it is the major active MMP determined by the resin method (FIG. 19c). Analyses here revealed the inadequacy of the widely used zymography assay for the purpose of identifying culprit MMPs in diseased tissues.

Methods for quantification of active MMP-8 and MMP-9 were developed using multiple-reaction monitoring (MRM), as described below. Data is shown in Table 5-1.

TABLE 5-1
Peptides and internal standard selected for MRM.

| Protein | Peptide Sequence | Precursor Ion | Product Ions |
|---|---|---|---|
| MMP-8 | CGVPDSGDFLLTPGSPK | 873.92 [M + 2H]²⁺ | 935.36 [M + H]⁺, |
| | | | 1074.58 [M + H]⁺, |
| | | | 959.56 [M + H]⁺ |
| MMP-9 | AFAVWGEVAPLTFTR | 832.94 [M + 2H]²⁺ | 1033.57 [M + H]⁺, |
| | | | 1090.59 [M + H]⁺, |
| | | | 734.42 [M + H]⁺ |
| | | 555.63 [M + 3H]³⁺ | 1090.59 [M + H]⁺, |
| | | | 1033.57 [M + H]⁺, |
| | | | 734.42 [M + H]⁺ |
| Yeast | NVNDVIAPAFVK | 643.86 [M + 2H]²⁺ | 632.38 [M + H]⁺, |
| enolase | | | 844.53 [M + H]⁺, |
| | | | 561.34 [M + H]⁺ |

It is noted that active MMP-8 and MMP-9 are present in the wounds of both wild-type and diabetic mice, except that MMP-9 is elevated at statistically significant levels only in diabetic wounds (FIG. 19c and Table 5-2).

TABLE 5-2
Concentrations of active MMP-8 and MMP-9 in wound tissues.

| | |
|---|---|
| Active MMP-8 | Active MMP-9 |
| (fmole/mg tissue) | (fmole/mg tissue) |

| Day | wild-type | db/db | wild-type | db/db |
|---|---|---|---|---|
| 0 | 17.6 ± 0.1 | 19.5 ± 1.5 | 0 | 0 |
| 1 | 18.3 ± 0.6 | 18.2 ± 0.6 | 17.9 ± 0.3 | 13.4 ± 4.5 |
| 3 | 17.8 ± 0.1 | 21.6 ± 3.4 | 18.1 ± 0.4 | 25.6 ± 6.5 |
| 7 | 17.8 ± 0.3 | 25.4 ± 0.7 | 20.4 ± 0.7 | 39.4 ± 13.7 |
| 10 | 21.1 ± 1.8 | 26.1 ± 3.1 | 26.3 ± 6.2 | 29.3 ± 2.3 |
| 14 | 17.9 ± 0.4 | 20.0 ± 2.3 | 19.2 ± 1.8 | 26.9 ± 9.1 |

Apoptosis is essential for normal wound repair. It regulates the removal of inflammatory cells and the conversion of granulation tissue into scar tissue. However, apoptosis is increased in diabetic wounds, which is likely to be instigated by the elevated levels of active MMP-9. This deregulated apoptosis leads to delayed wound healing in diabetes. Gutiérrrez-Fernandez et al. reported that MMP-8 is involved in healing of skin wounds (FASEB J 21 (10), 2580-2591 (2007)). Detection of active MMP-8 in the wounds of both wild-type and diabetic mice is likely a reflection of the effort by the tissue in healing.

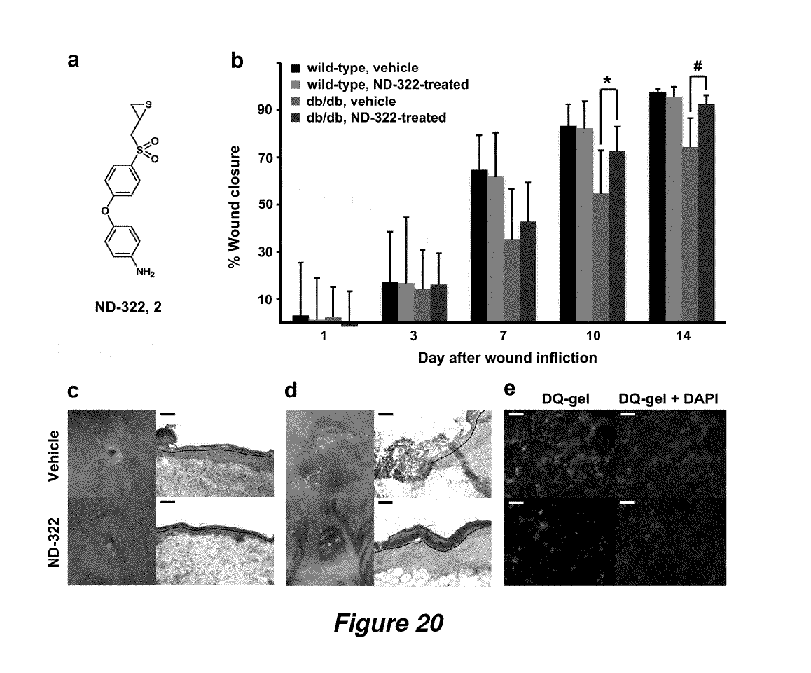

It was determined that MMP-9 plays a detrimental effect on diabetic wound healing. The inventors have worked on a class of selective thiirane MMP inhibitors, which are distinct from the commonly used broad-spectrum hydroxamate inhibitors, of which a library of a few hundred compounds has been prepared (Lee et al., ACS Med. Chem. Lett. 3 (6), 490-495 (2012)). This inhibitor class shows selectivity in targeting MMPs because of its unique mechanism of action, which involves ring-opening of the thiirane ring and generation of a thiolate at the active site. The effect of inhibitor 2 (also known as ND-322, FIG. 20a) was assessed. ND-322 exhibits selectivity in inhibition toward MMP-2, MMP-9, and MMP-14, on wound healing as a function of time as percentage of the initial wound area (FIG. 20b). Therefore, ND-322 inhibits selectively active MMP-9 found upregulated in diabetic wounds, while sparing MMP-8. The activity of MMP-9 is an impediment to healing of diabetic wounds and MMP-8 is necessary for wound repair, accordingly treatment with ND-322 can accelerate wound healing.

Differences in wound closure in diabetic and wild-type mice treated with vehicle were statistically significant on days 7, 10, and 14 (day 7: 35±21% vs. 65±15%, p<0.00001; day 10: 53±19% vs. 83±9%, p<0.0005; day 14: 74±12% vs. 98±1%, p<0.005). Wounds in wild-type mice were essentially healed on day 14 (FIG. 20c, top left), while those in diabetic mice lagged behind significantly (FIG. 20d, top left). Hematoxylin-eosin (H&E) staining revealed that wild-type mice showed complete re-epithelialization on day 14 (FIG. 20c, top right), whereas diabetic mice treated with vehicle had partial re-epithelialization (FIG. 20d, top right).

Topical treatment with ND-322 (FIG. 20a) of wounds in wild-type mice did not accelerate healing, compared to vehicle-treatment (day 7: 62±19% vs. 65±15%, n=21; day 10: 82±11% vs. 83±9%, n=14; day 14: 96±4% vs. 98±1%, n=7; p>0.25, FIG. 20b). Because active MMP-9 is not up-regulated in wounds of wild-type mice, treatment with an MMP-9 inhibitor does not appear to have any beneficial effect on wound healing in wild-type animals.

In contrast, topical treatment with ND-322 of wounds in diabetic mice accelerated wound healing (FIG. 20b). On days 1, 3, and 7, wound closures in ND-322-treated and vehicle-treated diabetic mice were not statistically significant (p>0.2, n=35, 28, and 21 on days 1, 3, and 7, respectively). On days 10 and 14, wound closure was significantly greater in ND-322-treated diabetic mice than in vehicle-treated diabetic mice (day 10: 70±16% vs. 53±19%, p<0.05, n=14; day 14: 92±4% vs. 74±12%, p<0.01, n=7). Remarkably, the extent of wound healing of diabetic mice treated with ND-322 on day 14 was comparable to that of wild-type mice (92±4% vs. 96±4%, p>0.14, n=7). Not only was wound healing in ND-322-treated diabetic mice more rapid, it also entailed complete re-epithelialization (FIG. 20d, bottom right); as is true for wild-type vehicle-treated wounds (FIG. 20c, top right) and wild-type ND-322-treated wounds (FIG. 20c, bottom right).

In-situ zymography, a technique that detects active MMPs localized in tissues and that is limited by the scarcity of substrates, showed considerable gelatinase activity in wound tissues of diabetic mice treated with vehicle (FIG. 20e, top left), which was significantly decreased on treatment with ND-322 (FIG. 20e, bottom left). Nuclei, as visualized with DAPI in vehicle-treated mice, were comparable to those in ND-322-treated animals (FIG. 20e, top right and bottom right).

In the present study, a novel resin was used for identification of active MMP-8 and MMP-9 in both diabetic and non-diabetic wounds, except the levels of the latter were elevated at statistically significant levels only in diabetic wounds. Identifying that MMP-9 was detrimental to healing of diabetic wounds, but that MMP-8 likely played a beneficial effect, MMP-9 was selectively inhibited by the use of ND-322. The diabetic wounds healed more rapidly in a process that involved re-epithelialization of the wounds, as is the case for the non-diabetic wounds in wild-type mice.

This example reveals the beneficial effect of selective inhibition of MMP-9 in healing of diabetic wounds, an enzyme that is upregulated. Other related examples and techniques include those described in U.S. Patent Application No. 61/522,554 filed Aug. 11, 2011. Whereas the use of the selective inhibitor ND-322 does not show any effect on non-diabetic wounds, neither detrimental nor beneficial, it is intriguing that the use of the broad-spectrum MMP inhibitor illomastat (also known as GM-6001) in non-diabetic wounds in rats, pigs, and humans exhibited significant deleterious effects, such as delayed wound closure and diminished epithelialization (Mirastschijski et al., Exp. Cell Res. 299 (2), 465-475 (2004); Agren, Arch. Dermatol. Res. 291 (11), 583-590 (1999); Agren et al., Exp. Dermatol. 10 (5), 337-348 (2001)). These findings reveal that broad inhibition of the “good” and the “bad” MMPs at once does not bode well for the healing process. Clinical management of diabetic wounds presently involves merely debridement of the wound and attempts at keeping it clean and free of infection. Disclosed herein is the first pharmacological intervention in treatment of diabetic wounds. The treatment of diabetic wounds with a selective MMP-9 inhibitor therefore provides significant new therapies for intervention of this disease.

Synthesis and Formulation of ND-322.

ND-322 was synthesized as reported previously (Gooyit et al., J. Med. Chem. 54 (19), 6676-6690 (2011)) and was dissolved in saline at a concentration of 5.0 mg/mL. The dosing solution and the vehicle (saline) were sterilized by filtration through an Acrodisc syringe filter (Pall Life Sciences, Ann Arbor, Mich., USA, 0.2 μm, 13 mm diameter, PTFE membrane).

Animals.

Female diabetic db/db mice (n=70, BKS.Cg-Dock7^{m}+/+Lepr^{db}/J, 6-8 weeks old, 38-40 g body weight, Jackson Laboratory, Bar Harbor, Me., USA) and female wild-type mice (n=70, C57BL/6J, 6-8 weeks old, 18-20 g body weight, Jackson Laboratory, Bar Harbor, Me., USA) were used. All procedures were performed in accordance with the University of Notre Dame Institutional Animal Care and Use Committee. Mice were provided with Laboratory 5001 Rodent Diet (PMI, Richmond, Ind., USA) and water ad libitum. Animals were maintained in polycarbonate shoebox cages with hardwood bedding in a room under a 12:12 h light/dark cycle and at 72±2° F.

Excisional Diabetic Wound Model.

Single excisional wounds 8-mm in diameter were made with a biopsy punch (Miltex, York, Pa., USA) using aseptic technique in the shaved dorsal regions of female diabetic db/db mice and female wild-type mice under isoflurane anesthesia. The diabetic and wild-type mice were each divided into two groups (35 per group): one group was treated with 50 μL of ND-322 in saline (equivalent to 0.25 mg per wound) and the other group with 50 μL of saline (vehicle). Wounds were photographed and immediately covered with a sterile dressing (3M Tegaderm™ Transparent Dressing, Butler Schein Animal Health, Inc., Dublin, Ohio). Cellulose acetate collars were made in-house from expired films and mounted on the wild-type mice to prevent them from disturbing the wounds on their back. Topical treatment with either ND-322 or vehicle commenced one day after wounding and continued for 14 days. On days 1, 3, 7, 10, and 14, digital photographs of wounds were taken while animals were under isoflurane anesthesia. On the same days, 14 mice (n=7 treated with ND-322 and n=7 vehicle) were sacrificed for wound-tissue sampling. The wounds with minimal surrounding healthy tissue were excised and either flash-frozen in liquid nitrogen for protein expression profiling or embedded in optimal cutting temperature (OCT) compound (Tissue-Tek, Torrance, Calif., USA) followed by cryosectioning for histological assessment.

Digital images were analyzed for wound areas using the NIH ImageJ version 1.45 software. Photographs of each wound were taken using a digital camera (Olympus SP-800UZ, Center Valley, Pa., USA), which was statically mounted on a tripod at a fixed distance above the mouse wound. A ruler was included in the photographic frame to allow ImageJ calibration. The wound outline was defined from the photographic image and the ImageJ software calculated the wound area. Wound closure was expressed as the change in wound area relative to that from day 0.

Statistical Analysis.

Wound closures are expressed as mean±SD (n=35 on day 1; n=28 on day 3; n=21 on day 7; n=14 on day 10; n=7 on day 14). Wound closures and levels of MMP-8 and MMP-9 were analyzed using a paired Student t-test; p<0.05 was considered statistically significant.

Synthesis of MMP Inhibitor-Tethered Resin.

The resin (FIG. 19 compound 1) was synthesized in our laboratories in twelve synthetic steps according to previously reported procedures (Hesek et al., J. Org. Chem. 71 (16), 5848-5854 (2006)).

Gelatin Zymography.

To assess gelatinolytic activity, aliquots of the tissue extracts, containing 0.4 mg of protein, were subjected to affinity precipitation with gelatin-agarose beads. The bound gelatinases were released from the beads in 2% SDS, and the samples were analyzed by electrophoresis in a 10% gelatin zymogram gel, as previously described (Toth and Fridman, Methods Mol. Med. 57, 163-174 (2001)).

Histological Evaluation and In-Situ Gelatin Zymography.

Fresh wound tissue was cut, embedded in OCT compound, and cryosectioned at a thickness of 12-μm in preparation for hematoxylin-eosin (H&E) staining. Morphological assessment of re-epithelialization was performed on a Nikon Eclipse 90i Fluorescent Microscope (Nikon Instruments Inc., Melville, N.Y., USA) (Tkalcevic et al., Toxicol. Pathol. 37 (2), 183-192 (2009)). In situ gelatin zymography was performed as described (Oh et al., J. Neurosci. 19 (19), 8464-8475 (1999)). Briefly, unfixed cryostat sections (12-μm-thick) of wound tissues were incubated in a reaction buffer (50 mM TBS pH 7.6) containing DQ-gelatin conjugate (Molecular Probes, Eugene, Oreg., USA) at 37° C. for 6 h. After fixation in 4% paraformaldehyde in PBS, cells were counterstained with DAPI (Molecular Probes, Eugene, Oreg., USA) and the images were visualized by fluorescence microscopy.

MMP-Expression Profiling.

Wound tissues (10 mg) were weighed and homogenized in 100 μL of cold lysis buffer (25 mM Tris-HCl pH 7.5, 100 mM NaCl, lie; Nonidet P-40 and protease inhibitors, with the exception of metalloproteinase inhibitors). The tissue extracts were diluted with CB buffer and were analyzed for protein concentration by the BCA protein assay. To the tissue extracts, 100 μL of inhibitor-tethered resin 1 (FIG. 19a) was mixed at 4° C. for 18 hours. After centrifugation (15000 g, 1 min), the supernatant was removed, the resin beads were thoroughly washed with CB buffer and water, and the resin bound proteins were subjected to trypsin-digest.

The procedure for on-resin reduction, alkylation, and tryptic digestion was adapted from the Pierce In-Solution Tryptic Digestion Kit (Thermo Scientific, Rockford, Ill., USA). Briefly, proteins bound to the resin were treated with 100 mM dithiothreitol in HPLC grade water. To the sample tube containing the resin-bound proteins was added a volume of the dithiothreitol solution sufficient to cover completely the resin. The tube was incubated at 65° C. for 20 min, then allowed to cool to room temperature. Samples were then alkylated by adding 3 μL of 100 mM iodoacetamide in HPLC-grade water to the cooled tubes, followed by incubation in the dark at room temperature for 20 min. Samples were then enzymatically digested overnight at 37° C. with trypsin (2 μL of 0.1 μg/μL in 50 mM ammonium bicarbonate). Following trypsin digestion, samples were desalted using Millipore ZipTip® C18 (EMD Millipore Corp., Billerica. MA), as described in the User Guide for Reversed-Phase ZipTip Pipette Tips for Sample Preparation. Briefly, each ZipTip® was wetted with HPLC-grade acetonitrile, equilibrated with 0.1% trifluoroacetic acid (TFA) in HPLC-grade water, loaded with 4 μL of digested sample, washed with 0.1% TFA in water, and eluted with 0.1% TFA in 50:50 (v:v) acetonitrile:water. This procedure was repeated until ˜20 μL of the eluted solution had been collected in an autosampler vial.

A 2-μL aliquot of the ZipTip® cleaned peptide mixtures were then analyzed on a reversed phase Waters nanoACQUITY column (1.7 μm, BEH130 C18, 100 μm i.d.×100 μm, Waters Corp., Milford, Mass.) coupled to a Thermo-Finnegan LTQ Velos Orbitrap tandem mass spectrometer (Thermo Fisher Scientific, Waltham, Mass., USA). Samples were eluted at a flow of 1.2 μL/min with the following gradient program: t=0-5 min 99% A/1% B, t=5.1 min 85% A/15% B, t=50 min 40% A/60% B, t=55 min 15% A/85% B, t=55.1-65 min 99% A/1% B where A=97% water/3% acetonitrile with 0.1% formic acid and B=0.1% formic acid in acetonitrile. Peptides were ionized via a nanoelectrospray ionization source, and their mass spectra and collisionally induced dissociation fragmentation mass spectra were recorded using a linear ion trap mass analyzer (LTQ Velos). High resolution (60,000 resolving power), accurate mass spectra were recorded between m/z 395-2,000 in ˜1.2 sec on the orbitrap mass analyzer. While the next high-resolution mass spectrum was being acquired on the orbitrap, the LTQ Velos linear ion trap independently recorded CID fragmentation mass spectra of the 8 most abundant ions present in the previous orbitrap mass spectrum. During the course of a 60-min nanoUPLC/MS/MS run, this approach typically generated ˜3,000 high-resolution mass spectra and between 12,000-15,000 CID MS/MS spectra.

Thermo-Finnegan Proteome Discoverer 2.0 software (Thermo Fisher Scientific, Waltham, Mass., USA) was used to interface with the Mascot (Matrix Science, Boston, Mass., USA) protein database search engine. MS/MS spectral information was used by Mascot to search the SwissProt Protein database, and a decoy search was employed to establish a false discovery rate. Standard solutions of MMPs that had been digested and analyzed using the approach described herein then served as references by which all results from the tissue-derived samples would be directly compared.

Quantification of MMPs/ADAMs in Wound Tissues.

The ZipTip® samples were concentrated to dryness on a miVac concentrator (Genevac Ltd., Suffolk, UK) and the residue was resuspended in 12 μL of water containing 1% formic acid and internal standard (yeast enolase at a final concentration of 150 fmole/mg tissue) was added. A 2-1 μL aliquot of the sample was injected directly onto a nanoACQUITY UPLC C18 column (1.8 μm, 100 μm i.d.×100 mm, Waters Corp., Milford, Mass.). The mobile phase consisted of 12-min elution at 600 mL/min with 2% acetonitrile/0.1% formic acid/water, followed by a 60-min linear gradient to 35% acetonitrile/0.1% formic acid/water. Samples were analyzed on a ABSciex QTrap 5500 mass spectrometer (ABSciex, Framingham, Mass., USA) running in ion trap IDA mode coupled to a two-dimensional Eksignet Ultra NanoUPLC system, consisting of a nanoLC ultra 2D pump and a nanoLC AS-2 autosampler (Eksignet, Dublin, Calif., USA).

The mass spectrometer was operated in the positive electrospray ionization (ESI) mode. The following conditions were used: curtain gas: curtain gas 20 psi, ion spray voltage 2350 V, ion source gas 1 10 psi, declustering potential 100V, entrance potential 10V, collision cell exit potential 40V. Acquisition parameters were as described previously (Llarrull et al, J. Biol. Chem. 286, 38148-58 (2011)). MRM transitions were determined through the use of empirical MS/MS data obtained from the bottom-up proteomics analysis and through the use of in silico prediction, as described previously (Llarrull et al, J Biol Chem 286, 38148-58 (2011)). MMP-8 and MMP-9 were quantified using three product-ion transitions per peptide, with one as the ‘quantifier’ and two as the ‘qualifier’ transitions (Table 5-1). The quantifier MMP-8 specific tryptic peptide [CAm]CGVPDSGDFLLTPGSPK was observed and quantified for the transition m/z 873.92 (M+2H)²⁺→product ion m/z 935.36 (M+H)⁺ corresponding to the b10 ion. The MMP-9 specific tryptic peptide AFAVWGEVAPLTFTR observed at m/z 832.94 (M+2H)²⁺→product ion m/z 1033.57 (M+H)⁺ (y9) was used. Quantification of MMP-8 and MMP-9 was relative to the yeast enolase (internal standard) peptide NVNDVIAPAFVK at m/z 643.86 (M+2H)²⁺→product ion m/z 632.38 (M+H)⁺ (y6). Standard calibration curves of MMP-8 and MMP-9 were prepared in control mouse skin tissue at concentrations of 0.6, 6.0, 15, 30, 60, 150, 300, and 600 fmole/mg tissue. Concentrations in unknown samples were determined using peak area ratios relative to the internal standard and regression parameters calculated from the calibration curve standards. Levels of MMP-8 and MMP-9 are expressed as mean±SD (n=3) and analyzed for statistical significance with a Student t-test; p<0.05 was considered statistically significant.

### Example 6

### Pharmaceutical Dosage Forms

The following formulations illustrate representative pharmaceutical dosage forms that can be used for the therapeutic or prophylactic administration of a compound described herein, a compound specifically disclosed herein, a composition thereof (e.g., one containing an MMP inhibitor), or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Compound X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Compound X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Compound X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraban | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method of accelerating the healing process of a wound comprising:
administering to a mammal afflicted with a wound an effective amount of an MMP inhibitor, or a pharmaceutically acceptable salt thereof, wherein the MMP inhibitor is effective to accelerate the healing process of the wound.

2. The method of claim 1 wherein the MMP inhibitor is a collagenase inhibitor.

3. The method of claim 1 wherein the MMP inhibitor is a gelatinase inhibitor.

4. The method of claim 3 wherein the MMP inhibitor selectively inhibits MMP-9.

5. The method of claim 4 wherein the MMP inhibitor does not inhibit MMP-8.

6. The method of claim 5 wherein the structure of the MMP inhibitor comprises a methyl-thiirane group.

7. The method of claim 6 wherein the gelatinase inhibitor is a compound of Formula I: 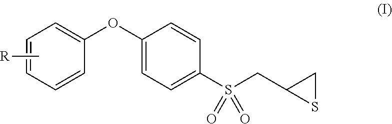
wherein R is H, OH, NH₂, NH-amino acid, or —X—(C═O)—R′ where X is O or NH, and R′ is alkyl, aryl, alkylaryl, amino, or alkoxy, where any alkyl, aryl, or amino is optionally substituted.

8. The method claim 1 wherein the effective amount of the inhibitor is about 0.01 mg to about 50 mg per day.

9. The method of claim 1 wherein the wound is an internal wound.

10. The method of claim 1 wherein the mammal is afflicted with diabetes.

11. The method of claim 1 wherein the inhibitor is applied topically.

12. The method of claim 1 wherein the mammal is afflicted with diabetes.

13. The method of claim 9 wherein the inhibitor is administered to the wound in an ointment composition.

14. The method of claim 1 wherein said administration of the inhibitor is systemic.

15. A method of inhibiting the progression of a wound associated disease state characterized by elevated levels of matrix metalloproteinases comprising:
administering to a mammal afflicted with said wound an effective amount of an MMP inhibitor, or a pharmaceutically acceptable salt thereof, effective to inhibit the progression of the wound in the mammal.

16. A method for enhancing the rate of repair of a wound comprising:
administering to a mammal afflicted with a wound an effective amount of a MMP inhibitor, or a pharmaceutically acceptable salt thereof, wherein the rate of repair of the skin wound is enhanced compared to the rate of repair of a wound not receiving the MMP inhibitor.

17. A dressing or patch for a chronic skin wound comprising:
a gelatinase inhibitor, or a pharmaceutically acceptable salt thereof, and
a pharmaceutically acceptable diluent, excipient or carrier for a transdermal composition;
wherein the gelatinase inhibitor and the diluent, excipient or carrier are combined and incorporated into a dressing or a patch, which optionally includes a backing layer, and adhesive, or both.

18. The dressing or patch of claim 17 wherein the gelatinase inhibitor is a compound of Formula I: 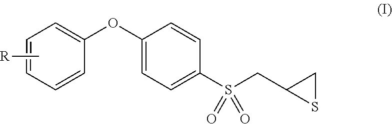
wherein R is H, OH, NH₂, NH-amino acid, or —X—(C═O)—R′ where X is O or NH, and R′ is alkyl, aryl, alkylaryl, amino, or alkoxy, where any alkyl, aryl, or amino is optionally substituted.
